(19) United States
(12) Patent Application Publication
(10) Pub. No.: US 2004/0062711 A1
(43) Pub. Date: Apr. 1, 2004

(54) Gastric emptying

(76) Inventor: Wolfram Meier-Augenstein, Dundee (GB)

Correspondence Address: WINSTON & STRAWN, PATENT DEPARTMENT, 1400 L STREET, N.W., WASHINGTON, DC, 20005-3502, US

(21) Appl. No.: 10/637,834

(22) Filed: Aug. 7, 2003

Related U.S. Application Data
(63) Continuation of application No. PCT/GB01/00528, filed on Feb. 7, 2002.

(30) Foreign Application Priority Data
Aug. 2, 2001 (GB) 0103097.2

(51) Int. Cl. A61K 49/00

(52) U.S. Cl. 424/9.1

(57) ABSTRACT

A stable solid food product that includes a carbohydrate, protein, fat, fiber and a source of ¹³C, is suitable for use in gastric emptying tests. Also, methods for preparing the food product and conducting a gastric emptying test utilizing the food product.

### CROSS-REFERENCE TO RELATED APPLICATION

This application is a continuation of International Application PCT/GB02/00528 filed Feb. 7, 2002, the entire content of which is expressly incorporated herein by reference thereto.

### BACKGROUND

The present invention relates to the provision of a solid food product, which is suitable for use in gastric emptying tests. A method of preparing the solid food product, which comprises a source of ¹³C, is provided, as well as the products themselves.

In those areas of medicine that deal with monitoring and therapy control, the current standard methods often involve exposure to ionizing radiation (radioisotopes; X-rays) or invasive procedures such as blood sampling, endoscopies or biopsies (which may require general anaesthesia). The risks associated with ionizing radiation limits the use of radioisotopes in terms of repeat studies and patient groups (children, pregnant women, breast feeding mothers and women of child-bearing potential). Invasive procedures are associated with small but real potential side effects such as inflammation and infection.

With the commercial availability of stable isotope ratio mass spectrometer (IRMS) systems, capable of highly precise measurement of small isotopic enrichment down to natural abundance level, stable isotope labelled compounds now offer a risk-free alternative to radioisotopes and radioisotope labelled compounds. The risk of uncertain long term side-effects associated with radioactive tracers is completely eliminated. There are also further cost benefits such as the avoidance of the indirect costs associated with storage and handling radioactive isotopes, which also reduces safety hazards for staff. The test can be made simple enough so that administration of the stable isotope tracer and collection of breath or urine samples may be carried by a technician or a nurse. This enables the non-specialist (e.g., a GP) to make use of this technique as no specialist qualification (such as that of a consultant) or special equipment is required for the test procedure as such.

The most easily utilized techniques of non-invasive testing involve probes that become oxidized to CO₂ during substrate metabolism. The CO₂ thus formed ultimately appears in exhaled breath, which is easily sampled. If it is known which carbon atom in the skeleton of the substrate is oxidized to CO₂, then a position specific ¹³C-labelled substrate can be used as a probe for a particular physiological process. After administration of the ¹³C-labelled probe, breath samples are taken at known intervals. These samples are measured by IRMS to detect the presence and amount of ¹³CO₂ in the breath.

Due to its non-invasive and uncomplicated nature, the ¹³CO₂-breath test can be carried out almost anywhere as it requires neither labor intensive procedures nor special equipment. It thus consumes only a fraction of the costs of endoscopies, biopsies, metabolic ratio tests from blood samples, histology, and bacterial cultures. Taking all its advantages into account, the ¹³CO₂-breath test provides a very attractive alternative to traditional invasive methods, especially when repeat testing or monitoring is required which are difficult using most current methods and often impossible as they would involve repeated exposure to ionizing radiation.

Probably the best known and most widely used is the ¹³C-urea breath test (¹³C-UBT) that detects the presence of the bacterium Helicobacter pylori in the stomach.

To avoid repeated exposure to ionizing radiation, attempts have been made to assess gastric emptying (GE) by means of a stable isotope based breath test as a non-radioactive alternative to radio-scintigraphy, the current standard procedure. Precise knowledge of this function is important because GE is rate-limiting for the absorption of meal constituents and pharmaceutical drugs by the more distal portions of the gut. Gastric emptying determines the rate of delivery to the small intestine where absorption occurs after hydrolysis by the mucosal brush border enzymes.

Delayed gastric emptying is associated with a variety of diseases such as irritable bowel syndrome (IBS), functional dyspepsia, AIDS, diabetes mellitus, gastroesophageal reflux disease (GERD) and heartburn. The underlying cause of diseases such as IBS and GERD often remains undiagnosed because the risks of endoscopy and radio-scintigraphy are considered to outweigh the benefit in these instances. Delayed GE can be treated by drugs promoting gastric motility but monitoring the success of such treatment requires repeated measurement of GE half-time and GE coefficient, a task for which radio-scintigraphy would be unsuitable.

Until recently, the only practical method of measuring the gastric emptying of solids by means of a ¹³C based breath test was the originally derived method of Ghoos et al [1]. Despite the fact that this test offered a valid alternative to radio-scintigraphy, the test itself has not assumed the widespread popularity, which one would have expected. The possible reasons for this lack of clinical acceptance may be due to a variety of factors. These might include: (1) the unpleasant taste and pungent aroma of octanoic acid, which leads to possible conflicts in compliance, especially where paediatric patients are concerned; (2) the test meal, a freshly prepared “omelette” made from separated egg white and egg yolk, lacks user-friendliness, i.e. its labor- and time-intensive preparation is not convenient in a clinical environment or a GP's surgery; and (3) the very nature of the test meal requires that it be freshly prepared for each subject.

Moreover, absorption and oxidation of fatty acids such as octanoic acid strongly depends on the individual physiology. Gastric emptying is therefore not the rate-limiting step in production and delivery of ¹³CO₂ to the breath thus impairing reproducibility and reliability (high coefficient of variation). Furthermore, octanoic acid is not suitable for assessing the gastric emptying of semi-solid or liquid meals due to its hydrophobic nature.

Accordingly, the present invention now obviates and/or mitigates one or more of the aforementioned disadvantages.

### SUMMARY OF THE INVENTION

The present invention relates to a method of preparing a solid food product comprising a source of ¹³C for use in a gastric emptying breath test. This method includes the steps of mixing a sugar syrup with butter or margarine, heating the mixture to form a homogeneous mixture; dissolving [1-¹³C] acetic acid or a salt thereof in the homogenous mixture; folding cereal into the mixture; forming the mixture into a desired shape; and baking this shape to a product.

The invention also relates to the solid food product that is obtained by the method. Preferably, the product is in the form of a small cake having a weight of 40 to 70 g.

The invention also relates to a method of performing a gastric emptying test in a subject which comprises administering to a subject in need of such test one of the solid food products disclosed herein, followed by periodically collecting and measuring ¹³CO₂ in breath samples from the subject to determine the extent of gastric emptying. Preferably, a background level of ¹³CO₂ is established in the subject prior to the administration of the solid food product.

### BRIEF DESCRIPTION OF THE DRAWING FIGURE

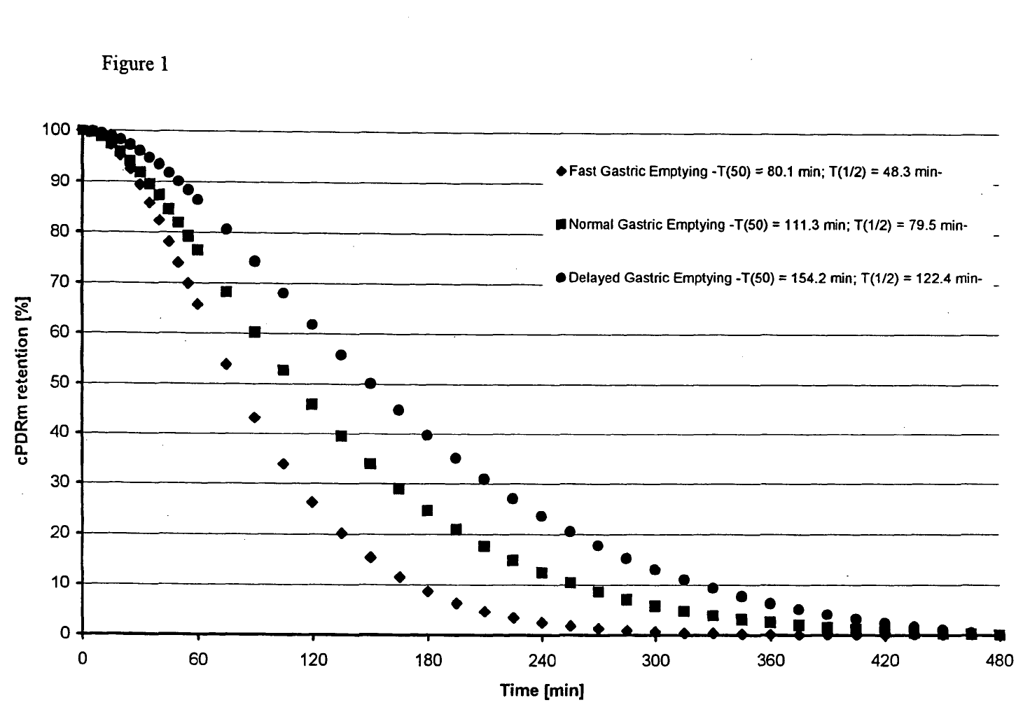

The present invention will now be further described by way of example as used in humans and with reference to FIG. 1 which shows the results of three subjects displaying, fast, normal and delayed gastric emptying times as determined using the test of the present invention.

### DETAILED DESCRIPTION OF THE PREFERRED EMBODIMENTS

In a first aspect there is provided a method of preparing a solid food product comprising a source of ¹³C for use in a gastric emptying breath test, comprising the steps of:

mixing a sugar syrup with butter/margarine and gently heating the mixture in order to melt the butter/margarine and form a homogenous mixture;

dissolving [1-¹³C] acetic acid or a salt thereof in the homogenous mixture;

folding cereal into the resulting mixture comprising [1-¹³C] acetic acid or salt thereof; and

forming the mixture comprising cereal into a desired shape and baking this at a low heat until firm.

The resulting solid food product, typically a flapjack or biscuit/cookie/cereal bar is particularly suited for use in a gastric emptying breath test because in addition to having a suitable source of ¹³C for assaying, the product also has sources of carbohydrate, protein, fat and fiber.

The sugar syrup is generally a sugar-cane derived syrup which is readily obtainable but it may also be a sugar-beet derived syrup. It is important to heat the syrup and butter/margarine (preferably unsalted) gently in order to prevent the butter from separating and, moreover, to ensure that the homogenous mixture is not too hot prior to the addition of the [1-¹³C] acetic acid or salt thereof. A suitable method of heating is to use the steam from boiling water in a method known typically as a “double boiler” method.

The [1-¹³C] acetic acid is generally added in its salt form as acetate and the most preferred form is sodium acetate.

¹³C acetate has a number of advantages for use as a probe. It is absorbed rapidly and completely via a Na⁺-, K⁺- and pH-independent transport mechanism in the small intestine and is not absorbed in the stomach; ¹³C-acetate is rapidly metabolized to ¹³CO₂ which is released in the breath and may easily be detected by known techniques. Moreover, it is readily soluble in the syrup and butter/margarine mixture and is evenly distributed throughout the resulting solid product. Importantly from a patient administration point of view is that sodium acetate is virtually tasteless such that the patient is not put off eating the product, unlike if octanoic acid is used.

The acetate is added in an amount such that each solid product comprises about 75-250 mg of ¹³C-acetate, more preferably 100-200 mg.

The cereal may be selected from wheat, barley, rice, oats or mixtures thereof. Oats are preferred and may typically be in the form of rolled oats, although other forms of oats, such as oatmeal or oat flakes may be used.

The mixture comprising the cereal may be formed into flapjacks, biscuits, cookies, or the like and is baked at a low heat in order to minimize any degradation, decomposition or loss of the [1-¹³C] acetic acid or salt thereof. Conveniently, the flapjacks/biscuits/cookies may be baked for 10-20 minutes at 100° C.-140° C. in a fan assisted oven. Generally speaking the flapjacks/biscuits/cookies/cereal bars should be baked until firm in texture and a light golden color.

It is to be understood that it is desirable that a solid food product for gastric emptying, should comprise all the basic food groups, i.e. protein, fat, carbohydrate and fiber. This is achieved with the present product. Ideally, the product should also have a high energy density. Typically the solid product should have a calorific value of 100-400 kcal, more conveniently 150-300 kcal. This may easily be achieved in the present process by appropriate control of the ingredients being added. Typically the amount of carbohydrate: protein: fat: fiber may be as follows:

| | |
|---|---|
| carbohydrate | 50-80% |
| protein | 5-20% |
| fat | 10-30% |
| fiber | 2-10% |

for a product weighing approximately 40-70 g.

In a particularly preferred embodiment the solid product is about 50-60 g in weight and comprises approximately 30-40 g carbohydrate; 3-5 g protein; 8-12 g fat; and 1.5-4 g fiber. The solid product of such formulation has a calorific value of approximately 220-250 kcal.

The present invention also provides solid products as described herein for use in gastric emptying tests.

The products of the present invention are particularly suitable for carrying out gastric emptying tests since (1) the ¹³C tracer becomes an integral part of the product itself; (2) the product comprises all the basic food groups in terms of its calorific value and nutritional composition; (3) the product has a high energy density and is of such a form as to allow quick consumption thus affording a well defined starting point of the test; (4) the product is equally appealing to a wide spectrum of age groups; and (5) the product is user-friendly in the sense that it can be pre-prepared in economically sized batches and stored for the short to medium term on the shelf without significant product deterioration.

As mentioned previously the solid product is used in a gastric emptying test. Specific details of such tests may be found in, for example, Schommartz et al [2]. In summary however, a test patient provides an initial breath sample in order to establish a natural background of ¹³CO₂. The patient then eats the solid product and breath samples are taken over a time course and the amount of ¹³CO₂ at each sample determined and equated with a degree of gastric emptying.

It is to be appreciated that the foregoing description relates in general to conducting gastric emptying tests on human subjects. However, such tests may also be conducted on animals such as horses, cows, sheep, dogs, cats and the like. The skilled reader would naturally understand that the size of solid product would require to be varied according to the size of animal being tested. Thus, for example, it may be expected that the size of solid product used to test a horse may be 2-4 times larger than that described above for human testing. Moreover, the solid product may be a third to half the size when used to test cats and dogs for example.

### EXAMPLES

The invention is further illustrated by the following examples of the most preferred embodiments.

### Example 1

### Preparation of Solid Product

The solid product is in the form of a “flapjack”, prepared in batches of 5, made from 100 g of sugar-cane derived syrup, 130 g of rolled oats and 50 g of unsalted butter. The butter is melted into the syrup using the steam of a hot water bath as heat source, thus not overheating the butter, which could cause the butter to separate. Once the butter and syrup have formed a homogenous mixture, 750 mg of [1-¹³C] acetic acid, as sodium salt (Cambridge Isotope Laboratories, Andover, Mass. 01810-5413, U.S.A.), are dissolved into this mixture. Once dissolution is complete, the rolled oats are carefully folded into this mixture. Five equal parts of approximately 55 g are weighed on to individual strips of baking foil and baked for 15 minutes at 120° C. in a fan-assisted oven.

Each individual flapjack has a calorific value of about 235 Kcal as calculated using tabulated values for the ingredients from reference (McCance & Widdowson's “The Composition of Food's” 1991, 5th Edition] and a composition of 35.6 g carbohydrate; 3.9 g protein; 9.2 g fat; and 2.5 g fiber.

### Example 2

### Use of Solid Product in Gastric Emptying Test

Breath Test Procedure

The breath test is carried out at preferably at 08.00 am after a 12 hours overnight fast with consumption of water allowed up until midnight. During the test, the subject remains in a seated and rested position. To establish the individual natural background signal of ¹³CO₂ in the breath, background breath samples are collected at 15 minutes before and immediately prior to ingestion of the test-meal. Breath samples are collected in standard gas sampling tubes (type Exetainer available from Isochem Ltd, Wokingham RG40 4RF, UK) by exhaling breath through a plastic straw. The test meal is consumed within 2 minutes and followed by 150 ml of water. Following test meal ingestion, breath samples are typically collected every 5 minutes during the first hour and every 15 minutes during the subsequent two hours. Preferably, duplicate breath samples are collected for each time point. The skilled reader would naturally understand that a smaller number of samples could be collected in conjunction with a data evaluation and modelling software.

In-Vitro Gastric Simulation

To assess the extent of marker retention in the solid phase of the meal and, hence, the suitability of the sodium acetate/flapjack combination as a test meal, a gastric simulation was carried out whereby 2 g aliquots of a sodium acetate/flapjack doped with 1 μCi of [1-¹⁴C] acetic acid, as sodium salt (specific activity: 5.3 mCi/mmol) was incubated with 10 ml of HCl (pH 2.3) at 37° C. Samples of the liquid phase were removed at intervals, centrifuged and aliquots of the supernatants removed for liquid scintillation. Results were then expressed as the percentage of the initial amount of radioactivity added, and compared to the results reported for the egg yolk omelette meal (according to the Ghoos et al procedure) doped with [1-¹⁴C] octanoic acid (cf. Table 1).

TABLE 1
¹⁴C Tracer retention in [%] during
in-vitro gastric stimulation at pH 2.3

| | Flapjack + | Egg yolk omelette + |
|---|---|---|
| Time[min] | sodium acetate | octanoic acid |
| 15 | 96.9 + 0.1 | |
| 30 | 95.3 + 0.1 | 97.8 + 0.7 |
| 60 | 95.2 + 0.9 | 95.7 + 0.5 |
| 120 | 92.3 + 1.8 | 91.0 + 1.4 |
| 180 | 87.4 + 1.9 | 86.8 + 2.3 |

As can be seen from Table 1, the results of the in-vitro gastric simulation show tracer retention for the flapjack/sodium acetate test meal similar to those reported for the omelette/octanoic acid test meal [1]. One can therefore conclude that the bulk of the tracer is an integral part of the test meal thus giving a true reflection of gastric emptying and the subsequent release of the tracer in the small intestine.

The advantage of the flapjack/acetate test meal (Table 2) is clearly illustrated by comparison of the mean gastric half-emptying time from 22 bona-fide healthy volunteers with published data for radio-scintigraphy and the octanoic acid breath test. Mean gastric half-emptying time was 108.7 min (SD 12 min; cv 11.05%). This value was not corrected for tracer retention.

TABLE 2
Gastric half-emptying times of solid meals:
data comparison of the three methods.

| Flapjack/Sodium acetate | | Egg Volk/Octanoic acid | | Radio-Scintigraphy | |
|---|---|---|---|---|---|
| | cv | | cv | | cv |
| t1/2 − 30 min | (%) | t1/2 − 66 min | (%) | t1/2 (min) | (%) |
| | | 72 ± 22.0 | 30.5 | 79.7 ± 20.0 | 25.1 |
| | | [1] | | [1] | |
| 78.6 ∀ 8.7 | 11.05 | 82 ± 19.4 | 23.7 | 93.5 | |
| | | [3] | | [5] | |
| | | 79 ± 24.4 | 30.9 | 86.0 ± 13.0 | 15.1 |
| | | [4] | | [6] | |

Corrected for the observed acetate retention time of 30 minutes [7; 8], the flapjack results were in excellent agreement with the published lag-phase corrected data for the octanoic acid breath test.

Furthermore, the flapjack results were also in excellent agreement with gastric half-emptying times reported for radio-scintigraphy. The most noticeable difference between the flapjack/acetate test and the egg yolk/octanoic acid test is the favorable coefficient of variation of 11.05% for the former as compared with 23.7 to 30.9% for the latter [1, 3, 4]. The cv of 11.05% for the flapjack/acetate test also compares favorably with cv's reported for radio-scintigraphy, ranging from 15.1 to 25.1% [1, 6].

Tests carried out on two GI patients are graphically presented in FIG. 1 to illustrate the potential of this new gastric emptying breath test to identify clinically significant gastric emptying rates outside the normal range. A Patient SJK underwent surgery to have a second exit crafted, connecting the stomach to the small intestine. The desired result of this operation, i.e. decreased residence time for food in the stomach, could be verified using the present gastric emptying breath test (FIG. 1 and Table 3). Compared to the control group, ▪, this patient's rate of gastric emptying ♦ is significantly increased. Patient PMT suffers from self-reported GI problems such as GERD and IBS. In this case, the gastric emptying breath test revealed significantly delayed gastric emptying ● (FIG. 1 and Table 1).

What is claimed is:

1. A method of preparing a solid food product comprising a source of ¹³C for use in a gastric emptying breath test, comprising the steps of:
mixing a sugar syrup with butter or margarine and gently heating the mixture in order to melt the butter or margarine and form a homogeneous mixture;
dissolving [1-¹³C] acetic acid or a salt thereof in the homogenous mixture;
folding cereal into the mixture that contains the dissolved [1-¹³C] acetic acid or salt thereof;
forming the mixture that includes the cereal into a desired shape; and
baking this shape to form a firm product.

2. The method according to claim 1 wherein the [1-¹³C] acetic acid is in its salt form.

3. The method according to claim 2 wherein the [1-¹³C] acetic acid is present as the sodium acetate salt.

4. The method according to claim 3 wherein the acetate salt is added in an amount such that the product comprises about 75-250 mg of ¹³C-acetate.

5. The method according to claim 1 wherein amounts of the sugar syrup; butter or margarine and cereal are controlled such that a 40 to 70 g amount of the product includes
50-80% carbohydrate;
5-20% protein;
10-30% fat; and
2-10% fiber.

6. The method according to claim 5 wherein the product has a weight of about 50-60 g and includes
30-40 g carbohydrate;
3-5 g protein;
8-12 g fat; and
1.5-4 g fiber.

7. A solid food product obtained by the method according to claim 1.

8. The product according to claim 7 wherein the [1-¹³C] acetic acid is in its salt form.

9. The product according to claim 8 wherein the [1-¹³C] acetic acid is present as the sodium acetate salt.

10. The product according to claim 9 wherein the acetate salt is present in an amount of about 75-250 mg.

11. The product according to claim 1 in the form of a small cake having a weight of 40 to 70 g.

12. The product according to claim 7 wherein amounts of the sugar syrup; butter or margarine and cereal are controlled such that the cake includes
50-80% carbohydrate;
5-20% protein;
10-30% fat; and
2-10% fiber.

13. The product according to claim 12 wherein the product has a weight of about 50-60 g and includes
30-40 g carbohydrate;
3-5 g protein;
8-12 g fat; and
1.5-4 g fiber.

14. A method of performing a gastric emptying test in a subject which comprises administering to a subject in need of such test the solid food product of claim 7 followed by periodically collecting and measuring ¹³CO₂ in breath samples from the subject to determine the extent of gastric emptying.

15. The method according to claim 14, wherein a background level of ¹³CO₂ is established in the subject prior to the administration of the solid food product.

16. A method of performing a gastric emptying test in a subject which comprises administering to a subject in need of such test the solid food product of claim 13 followed by periodically collecting and measuring ¹³CO₂ in breath samples from the subject to determine the extent of gastric emptying.

17. The method according to claim 15, wherein a background level of ¹³CO₂ is established in the subject prior to the administration of the solid food product.
